SEQUENCE LISTING (4 sequences)

SEQ ID NO: 1 - protein, 4 aa, Homo sp.
FWKT

SEQ ID NO: 2 - protein, 102 aa, Homo sapiens
SYRFKQGFRRVLLRPSRRVRSQEPTVGPPEKTEEEDEEEEDGEESREGGKEMNGRVSQITQGPGTSGQEVRPPSRVASKEQQLLPQEASTGEKSSTMRISYL

SEQ ID NO: 3 - protein, 49 aa, Homo sapiens
AFLSDNFKKSFQNVLCLVKVSGTDDGERSDSKQDKSRLNETTETQRTLL

SEQ ID NO: 4 - protein, 36 aa, Homo sapiens
GFLSDNFRQSFQKVLCLRKGSGAKDADATEPRPDRI

(19) United States
(12) Patent Application Publication
(10) Pub. No.: US 2025/0057913 A1
(43) Pub. Date: Feb. 20, 2025

(54) USE OF SOMATOSTATIN AGONIST IN THE TREATMENT OF SSTR3 EXPRESSING TUMORS

(71) Applicant: ITALFARMACO SPA, Milano (IT)

(72) Inventors: Daniela MODENA, Monza (IT); Giovanni SANDRONE, Novara (IT); Andrea STEVENAZZI, Milano (IT); Christian STEINKÜHLER, Roma (IT); Natalia S. PELLEGATA, München (DE); Stefan SCHULZ, Jena (DE)

(21) Appl. No.: 18/721,923

(22) PCT Filed: Dec. 22, 2022
(86) PCT No.: PCT/EP2022/087485

§ 371 Date: Jun. 20, 2024

(30) Foreign Application Priority Data
Dec. 24, 2021 (IT) 102021000032615

(51) Int. Cl. A61K 38/12; A61K 45/06; A61P 35/00

(52) U.S. Cl. CPC A61K 38/12; A61K 45/06; A61P 35/00; A61K 2121/00

(57) ABSTRACT

The present invention relates to the use of the compound of formula (I) or the pharmaceutically acceptable salts and/or solvates thereof in the treatment of SSTR3 expressing tumors, selected from non-functioning pituitary adenomas (NFPAs) or other neuroendocrine-related malignancies, selected from pancreatic tumors, pheochromocytomas, paragangliomas, lung carcinoids or breast cancer. More specifically, the present invention provides to the use of pharmaceutical compositions comprising the compound of formula (I) or the pharmaceutically acceptable salts and/or solvates thereof and at least one physiologically acceptable excipient in the treatment of a patient affected by SSTR3 expressing tumors, selected from non-functioning pituitary adenomas (NFPAs) or other neuroendocrine-related malignancies, selected from pancreatic tumors, pheochromocytomas, paragangliomas, lung carcinoids or breast cancer.

### BACKGROUND OF THE INVENTION

Non-functioning pituitary adenomas (NFPAs) are benign adenohypophyseal tumors not associated with clinical evidence of hormonal hypersecretion. NFPAs are mainly gonadotroph pituitary adenomas and account for approximately 35% (14-54%) of all pituitary tumors. Their prevalence is 7-41.3/100,000 population and standardized incidence rate is 0.65-2.34/100,000; the peak occurrence is from the fourth to the eighth decade^{1,2.}

NFPAs are often diagnosed at the occurrence of signs and symptoms of “mass effects” such as headaches, visual disorders and/or cranial nerve dysfunction caused by compression and lesions extending into the cavernous sinus and the sellar floor.³ Moreover, some cases are diagnosed incidentally through imaging studies performed for other purposes. Hypopituitarism and hyperprolactinemia, due to the compression of the normal anterior pituitary and to pituitary stalk deviation, respectively, can also be present.

Currently the standard first line therapies for most NFPAs are endoscopy or microscopy assisted trans-sphenoidal surgery and transcranial surgery, the second one used for predominantly suprasellar tumors.⁴

After surgical treatment NFPAs often progress, with regrowth rates of 15-66% in NFPA patients treated with surgery alone and 2-28% in those treated with surgery followed by radiotherapy.^{5,6} Moreover, the systematic use of radiotherapy is limited by its side effects.

Therefore, an adjuvant and/or alternative post-operative therapy is a relevant medical need. However, despite their frequency, no standard of care drug treatment is currently recommended for NFPAs or for GPAs (gonadotroph pituitary adenomas).^{7,8}

Three main classes of peptides have been studied for the treatment of NFPA: dopamine receptor 2 (DR2) agonists, somatostatin agonists (SSA) and gonadotropin-releasing hormone (GnRH) analogues. In addition, the use of temozolomide has been introduced in some centers as therapy for aggressive tumors.

The efficacy of DR2 agonists (cabergoline and bromocriptine) correlates with the expression of the receptor and some studies demonstrated efficacy only when the drugs are administered immediately after surgery.

No evidence of GnRH analogues efficacy has been demonstrated. Moreover, in some cases GnRH agonists exacerbated gonadotropin secretion with no change in tumor growth or induced pituitary apoplexy, when used as therapy for metastatic prostate carcinoma in patients bearing also gonadotroph adenoma.⁹

SSAs like Octreotide and Lanreotide, which bind to somatostatin receptor SSTR2 and to a lesser extent to SSTR5 and SSTR3, are effective in the treatment of secreting pituitary adenomas¹⁰⁻¹², but are poorly efficacious in NFPAs.¹³

Similarly, the pan-agonist Pasireotide, which binds to SSTR1, 2, 3, 5, showed only modest efficacy in a recent phase II clinical trial (NCT01283542—Evaluate the Efficacy and Safety of Pasireotide LAR (Long Acting Release) on the Treatment of Patients With Clinically Non-Functioning Pituitary Adenoma—Passion I) with only 16.7% of patients reaching a tumor size reduction at least 20%.^{8,14}

There is therefore the need of new and effective pharmacological treatment that can be effectively used for the patients affected by NFPAs or by other neuroendocrine-related malignancies, with low side effects.

### BRIEF DESCRIPTION OF THE FIGURES

FIG. 1. Structure of ITF2984, Pasireotide and Octreotide

Represents the structures of the somatostatin analogues (SSA) evaluated in the present study: (A) ITF2984, (B) Pasireotide and (C) Octreotide.

FIG. 2. Best ZDOCK score pose of SRIF-14 (blue tube) superposed to SSTR3 and SSTR2 “active” conformers. EL4 5 loop reduces channel entrance in SSTR2 (red ribbon), whereas does not interfere in SSTR3. Top and bottom pictures are front and top point of view, respectively. Top right panel ligand Trp8 side chain pocket (top right side), Bottom right panel: Lys8 in the inner space: ζ Nitrogen atom lies close to tertiary amine of k-opiod agonist MP1104.

FIG. 3. Detail of Phe-Trp-Lys-Thr motif of SRIF-14 in best scored pose and generic sketch summarizing reference values of backbone torsions of I,I′,II and II′ β-turns. Measured dihedral angles ϕ, ψ of Trp-. Lys dyad are in qualitative agreement with ideal β turn type II′.

FIG. 4. Inhibition of GHRH-stimulated GH release in vitro using primary cultures of rat anterior pituitary cells.

The graph represents the inhibition of GHRH-stimulated GH release in vitro using primary cultures of rat anterior pituitary cells induced by Octretide, Pasireotide and ITF2984. Results are expressed as mean values±SD of 3 experiments.

FIG. 5. ITF2984-induced internalization of SSTR3 in HEK293 cells. (A, B)

HEK293 cells stably expressing wild-type h SSTR3 were treated with 1 μM or 10 μM SST14, Octreotide, Pasireotide or ITF2984 for 30 min. Cells were then fixed, stained with the anti-HA antibody and examined by confocal microscopy. Shown are representative images from one of at least three independent experiments performed in duplicate.

FIG. 6 Internalization of SSTR3, following treatment with SST28, Pasireotide, ITF2984 and Octreotide in U2OS cells.

FIG. 6 shows the internalization of SSTR3, following treatment with SST28, Pasireotide, ITF2984 and Octreotide (Mean of internalized fluorescence in Arbitrary Units (AU)).

FIG. 7. ITF2984-selective SSTR3 phosphorylation in HEK293 transfected cells.

(A) Schematic representation of the human SSTR3 receptor indicating all potential phosphate acceptor sites within the carboxyl-terminal tail. Epitopes of the phosphosite-specific antibodies are marked (-P).

(B, C) HEK293 cells stably expressing wild-type hSSTR3, were either not exposed or exposed to 10 μM SST14, Octreotide, Pasireotide or ITF2984 in concentrations ranging from 10⁻¹² to 10⁻⁵ M (B); 10 μM SST14, Octreotide, ITF2984 or Pasireotide in concentrations ranging from 10⁻¹² to 10⁻⁵ M (C) for 10 min at 37° C. The levels of phosphorylated SST3 receptor were then determined using the phosphosite-specific anti-pS337/pT341 or anti-pT348.

FIG. 8. Agonists-selective SSTR2 phosphorylation in HEK293 transfected cells.

(A) Schematic representation of the human SSTR2 receptor indicating all potential phosphate acceptor sites within the carboxyl-terminal tail. Epitopes of the phosphosite-specific antibodies are marked (-P). (B, C) HEK293 cells stably expressing wild-type hSSTR2, were either not exposed or exposed to SST14 (=SS14), Octreotide, Pasireotide or ITF2984 at 10 or 1 μM concentrations for 10 min at 37° C. The levels of phosphorylated SSTR2 receptor were then determined using the phosphosite-specific anti-pS341/pS343, pT353/T354, pT356/T359 antibodies. Blots were subsequently stripped and reprobed with UMB1 antibody to confirm equal loading of the gels. Pasireotide and ITF2984 induce a selective phosphorylation of S341/S343. In contrast, SST14 and Octreotide induce a full phosphorylation of SSTR2. Blots shown are representative of three independent experiments. The positions of molecular mass markers are indicated on the left (in kDa).

FIG. 9—Agonist-selective SSTR5 phosphorylation in HEK293 transfected cells. (A) Schematic representation of the human SSTR5 receptor indicating all potential phosphate acceptor site within the carboxyl-terminal tail. Epitope of the phosphosite-specific antibodies are marked (-P) . . . (B) HEK293 cells stably expressing wild-type hSSTR5, were either not exposed or exposed to SS14, Octreotide, Pasireotide or ITF2984 at the indicated concentrations for 10 min at 37° C. The ability of SST14, Octreotide, Pasireotide and ITF2984 to induce phosphorylation of the SSTR5 was tested by Western blot using the phosphosite specific anti-pT333 antibody. ITF2984 induces a partial phosphorylation of SSTR5. Pasireotide was more potent than ITF2984 and octreotide. (C) Blots were subsequently stripped and reprobed with anti-HA antibody to confirm equal loading of the gels. Blots shown are representative of three independent experiments. The positions of molecular mass markers are indicated on the left (in kDa).

FIG. 10. Agonist-mediated G protein-signalling of SSTR3 in HEK293 transfected cells.

The ability of SST14, Octreotide, Pasireotide and ITF2984 to activate GIRK2 channels via the SSTR3 was tested using a fluorescence membrane potential assay. The concentrations used are indicated. ITF2984 induced a strong G-protein signaling at SSTR3—about 5 times more potent than Octreotide or Pasireotide. Data points represent mean±S.E.M.

FIG. 11. Analysis of G protein-signalling in mouse AtT-20 cells using a fluorescence-based membrane potential assay.

(A) The ability of Octreotide to activate endogenous GIRK channels in wild type AtT-20 cells via the endogenously expressed SSTR2 and SSTR5 receptors (black) or in exogenously expressed human SSTR3 receptors (grey) was tested. (B) The ability of ITF2984 to activate endogenous GIRK channels in wild type AtT-20 cells via the endogenously expressed SSTR2 and SSTR5 receptors (black) or in exogenously expressed human SSTR3 receptors (grey) was tested. Expression of SSTR3 resulted in a leftward shift of the dose-response curve.

FIG. 12. Changes in tumor volume in rats treated with ITF2984 or placebo.

MENX-affected rats at the age of 5.5 months were injected with ITF2984 1× every 14 days at the indicated dose (12.5 mg/kg of body weight). MRI was performed every 14 days and the tumor volume was normalized against the volume at day 0. Male and female rat tumors are shown separately. Data presented are the mean±SEM. #, not significant; *, p-value <0.05; **, p-value <0.001.

FIG. 13. Proliferation of NFPAs in rats treated with ITF2984 or placebo.

(A, B) Number of Ki67-positive cells per 100.000 μm2 in tumors of rats belonging to the 2 groups (ITF2984-treated and control) and both sexes. Shown is the mean±SEM. *, p-value <0.05; **, p-value <0.001.

FIG. 14. Sstr gene expression in male and female rat bearing NFPAs treated with ITF2984 or placebo.

Absolute quantification of mRNA copy number/cell for SSTR1,2,3,5 genes in placebo-treated control rats of both sexes. (B) Relative expression of the Sstr1,2,3,5 genes in the ITF2984 treatment group compared to the control group, arbitrarily set to 100%. Shown is the average±SEM. *, p-value <0.05.

FIG. 15. Cytotoxic effect of increasing concentration (from 20 to 2560 nM) of ITF2984 on NT-3 cell line spheroids treated for 18 days, Pictures were always taken from the same well at days 0, 4, 7, 11, 14, 18. Day 0 corresponds to the first day of treatment.

FIG. 16. Cytotoxic effect of increasing concentration (from 20 to 2560 nM) of Octreotide on NT-3 cell line spheroids treated for 18 days, Pictures were always taken from the same well at days 0, 4, 7, 11, 14, 18. Day 0 corresponds to the first day of treatment.

FIG. 17. Cytotoxic effect of increasing concentration (from 20 to 2560 nM) of Pasireotide on NT-3 cell line spheroids treated for 18 days, Pictures were always taken from the same well at days 0, 4, 7, 11, 14, 18. Day 0 corresponds to the first day of treatment.

### DEFINITIONS

Unless otherwise defined, all terms of art, notations and other scientific terminology used herein are intended to have the meanings commonly understood by those persons skilled in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “physiologically acceptable excipient” herein refers to a substance devoid of any pharmacological effect of its own and which does not produce adverse reactions when administered to a mammal, preferably a human being. Physiologically acceptable excipients are well known in the art and are described, for example, in the Handbook of Pharmaceutical Excipients, sixth edition (2009), which has been incorporated herein for reference purposes.

The term “salts and/or pharmaceutically acceptable derivatives” herein refers to those salts or derivatives which have the biological effectiveness and the properties of the salified or derivate compound and which do not produce adverse reactions when administered to a mammal, preferably a human being. The pharmaceutically acceptable salts may be inorganic or organic salts; examples of pharmaceutically acceptable salts include, but are not limited to: carbonate, hydrochloride, hydrobromide, sulphate, hydrogen sulphate, citrate, maleate, fumarate, trifluoroacetate, 2-naphthalenesulfonate, and paratoluenesulfonate. More information on the pharmaceutically acceptable salts can be found in the Handbook of pharmaceutical salts¹⁵, incorporated herein for reference purposes. The pharmaceutically acceptable derivatives include esters, ethers, and N-oxides.

The term “simultaneous, separate, or sequential use” herein refers to the simultaneous administration of the first and second compound, or administration in such a way that the two compounds act in the patient's body simultaneously or administration of one compound after the other compound so as to provide a therapeutic effect. In some embodiments, the compounds are taken with a meal. In other embodiments, the compounds are taken after a meal, for example 30 minutes or 60 minutes after the meal. In some embodiments, a compound is administered to a patient for a period of time, followed by administration of the other compound.

The terms “comprising”, “having”, “including” and “containing” should be understood as ‘open’ terms (i.e. meaning “including, but not limited to”) and should also be deemed a support for terms such as “consist essentially of”, “consisting essentially of”, “consist of”, or “consisting of”.

The abbreviation “NFPA” in the present invention means “non-functioning pituitary adenomas”.

With the term “SSA” in the present invention we intend somatostatin analogs.

With the term “PAN-agonist” in the present invention we intend agonist of somatostatin receptors (SSTR1, 2, 3 and 5).

“Lung carcinoid tumor” is a type of cancerous tumor made up of neuroendocrine cells. These cells are found throughout the body, including the lungs. They are considered endocrine cells because both produce and secrete hormones or hormone-like substances.

“Pheochromocytomas” is a rare tumor of the adrenal medulla composed of chromaffin cells, also known as pheochromocytes. When a tumor composed of the same cells as a pheochromocytoma develops outside the adrenal gland, it is referred to as a “paragangliomas”. These neuroendocrine tumors are capable of producing and releasing massive amounts of catecholamines, metanephrines, or methoxytyramine, which result in the most common symptoms, including hypertension (high blood pressure), tachycardia (fast heart rate), and diaphoresis (sweating). However, not all of these tumors will secrete catecholamines. Those that do not are referred to as biochemically silent, and are predominantly located in the head and neck.

### Description of the Invention

As it will be disclosed in details in the Experimental Section, the present inventors has surprisingly found that ITF2984, a novel cyclic SSA pan-agonist hexapeptide with an elevated binding affinity for SSTR3 and improved properties versus first-generation SSAs, is effective in the treatment of NFPAs in the MENX (homozygous mutant) NFPA rat model, which closely resembles the human counterpart^{16,17}.

During the molecular characterization of ITF2984, the inventors found that while structurally analogous to other pan-agonists, such as Pasireotide and Octreotide, ITF2984 has a higher affinity for SSTR3 as compared to known molecules. Molecular modeling revealed that a higher β-II′ turn probability in ITF2984 correlated with higher SSTR3 affinity, thus providing a structural rationale for the unique selectivity pattern of this molecule.

In particular, the in-vitro results obtained surprisingly show that ITF2984 induced SSTR3 internalization and phosphorylation more effectively than Pasireotide or Octreotide in two different cell lines, as well as GIRK activation in a pharmacologically relevant concentration range. Based on these data, ITF2984 can therefore be considered a full agonist of the SSTR3 receptor, promoting receptor internalization.

Several studies report that SSTR3 is frequently and strongly expressed in gonadotroph adenomas, while SSTR2 is expressed only in a small number of patients and SSTR5 only exceptionally.^{3,8,18,19}

Moreover, besides pituitary adenomas, recent studies suggest an elevated SSTR3 expression in diverse neuroendocrine-related malignancies, such as pancreatic tumors²⁰, pheochromocytomas, paragangliomas²¹, lung carcinoids²² and breast cancer.²³

Non-functioning pituitary adenomas (NFPAs) and other neuroendocrine-related malignancies, such as pancreatic tumors²⁰, pheochromocytomas, paragangliomas²¹, lung carcinoids²² and breast cancer²³ are therefore herein considered as SSTR3 expressing tumors.

SSTR3 activation by somatostatin (SST) and SSA induces cytostatic and cytotoxic effects by interfering with mitogenic pathways through activation of protein tyrosine phosphatases and subsequent inactivation of Raf1 and MAPK. In addition, SSTR3 engagement was proposed to induce apoptosis, through p53 and caspase activation. SSTR3 targeting also inhibits endothelial cell proliferation and consequently neo-angiogenesis.^{10,24-26} The development of a new SSA that recognizes and activates SSTR3 is therefore a potentially promising strategy for the treatment of NFPAs, due to the fact that:
1) NFPAs mainly express SSTR3, which is maintained also after radiotherapy³ and
2) the response of pituitary adenomas to SSAs depends on the expression of specific SSTR subtypes, as seen for SSTR2 in GH-secreting adenomas.²⁷

This has been confirmed by the results obtained in the in-vivo studies reported in the Experimental section, that clearly demonstrate that ITF2984 effectively suppresses the tumor growth in mice and induces a strong reduction in NFPA cells proliferation.

Consistent with the receptor affinity profile found and with the in vitro data obtained, ITF2984 showed therefore a selective antitumor activity.

This antitumor activity went along with a decrease in the proliferation index (KI67 positivity) of ITF2984-treated tumors. In addition, ITF2984 selectively induced SSTR3 mRNA expression in female rats, suggesting a compensatory upregulation.

The data obtained are therefore in line with an in vivo engagement of SSTR3 and a predominantly SSTR3-driven antitumor activity of ITF2984 in this model and provide an in vivo proof of concept for the potential clinical use of ITF2984 in NFPAs and other SSTR3-driven diseases, such as SSTR3 expressing tumors (i.e. pancreatic tumors, pheochromocytomas, paragangliomas, lung and breast carcinomas) and ciliopathies.

Recently, the activities of Pasireotide and Octreotide in the MENX model were reported²⁸ and the inventors compare those data with the results on ITF2984 reported herein. In the published report Pasireotide was shown to have a higher antitumor activity than Octreotide, that was evident on both female and male rats, with a trend towards a higher activity in females. That enhanced activity was ascribed to an involvement of the SSTR3 receptor.

We notice that these data differ from our observations using ITF2984. In fact, ITF2984 showed a significant radiologic tumor response and a compensatory upregulation of SSTR3 mRNA only in female rats, while no significant antitumor effect was observed in male rats with lower SSTR3 levels. We interpret these data in terms of a more selective activity of ITF2984, predominantly, if not exclusively, mediated by SSTR3 engagement under the tested experimental conditions.

It has also been observed that the animals treated show no signs of discomfort at the end of the treatment. The safety of ITF2984 has also been confirmed in the preclinical safety studies, in the two Phase I clinical trials in normal healthy volunteers and in a Phase II trial in acromegaly patients, showing efficacy at tolerated doses (39, 40).

It is therefore an embodiment of the present invention the use of the compound of formula (I):

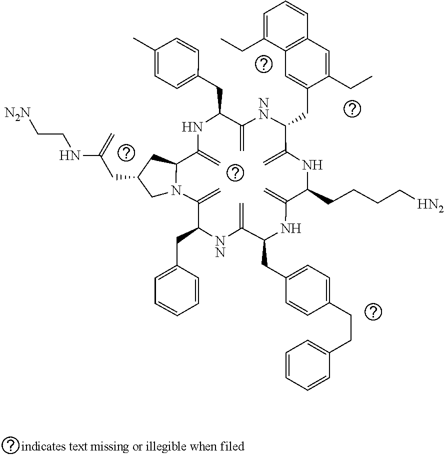

or of the pharmaceutically acceptable salts and/or solvates thereof in the treatment of SSTR3 expressing tumors.

According to a preferred embodiment said SSTR3 expressing tumors are non-functioning pituitary adenomas (NFPAs) or neuroendocrine-related malignancies, selected from pancreatic tumors, pheochromocytomas, paragangliomas, lung carcinoids or breast cancer. Preferably said pharmaceutical acceptable salts and/or solvates are pamoate, diacetate or trifluoroacetate.

The IUPAC name of the compound of formula (I) is (3S,6R,9S,12S,15S,19R,20aS)-9-(4-aminobutyl)-15-benzyl-12-(4-(benzyloxy)benzyl)-6-((3,8-dimethoxynaphthalen-2-yl)methyl)-3-(4-hydroxybenzyl)-1,4,7,10,13,16-hexaoxoicosahydropyrrolo[1,2-a][1,4,7,10,13,16]hexaazacyclooctadecin-19-yl (2-aminoethyl)carbamate or Cyclo[4(R)-[N-(2-aminoethyl)carbamoyloxy]-L-prolyl-L-tyrosyl-D-3,8-dimethoxynaphthylalanyl-L-lysyl-(4-O-benzyl)-L-tyrosyl-L-phenylalanyl].

The compound of formula (I) is herein indicated also as ITF2984 and was already disclosed in the international patent application WO2009/071460A2.

According to a preferred embodiment of the present invention, the compound of formula (I) or the pharmaceutically acceptable salts and/or solvates thereof is administered to a patient on a daily basis. Preferably said patient is a human.

According to a further preferred embodiment, the compound of formula (I) or the pharmaceutically acceptable salts and/or solvates thereof is administered to a patient in an amount ranging from 0.5 to 5 mg/die, preferably from 0.2 to 2.5 mg/die, more preferably from 0.1 to 2 mg/die. Preferably the compound of formula (I) or the pharmaceutically acceptable salts and/or solvates thereof is administered to a patient in a quantity of 0.1 mg twice a day, preferably 0.5 mg twice a day, more preferably 5 mg once a day.

Preferably, the compound of formula (I) or the pharmaceutically acceptable salts and/or solvates thereof is administered by oral, sublingual, rectal, intravascular, intravenous, subcutaneous route, preferably by oral route. According to a further preferred embodiment, the compound of formula (I) or the pharmaceutically acceptable salts and/or solvates thereof is administered to a patient in the form of a pharmaceutical composition containing the same together with at least one physiologically acceptable excipient.

Preferably, said pharmaceutical composition is administered by oral, sublingual, rectal, intravascular, intravenous, subcutaneous route, preferably by intravenous route.

Preferably, said pharmaceutical composition is in a solid or a liquid form. More preferably, said solid form is selected from powder, tablet, granulate, aggregate, compressed or coated pill, hard or gelatine capsule; said liquid form is a suspension, a syrup or a liquid for injection.

According to a preferred embodiment of the present invention, the compound of formula (I) or the pharmaceutically acceptable salts and/or solvates thereof is administered to a patient in combination with at least one active principle.

Preferably said at least one active principle is selected from a secretagogue of the insulin, a promoter of the insulin secretion, an insulin sensitizer, a low insulin dose, an agent with dopamine receptor 2 agonism, an agent having anti-angiogenic effects or a chemiotherapic agent.

Preferably said agent with an insulin sensitizer activity is metformin which decreases gluconeogenesis in the liver.

Preferably said agents with a secretagogues of the insulin are sulfonylureas or incretin-based drugs, selected from vindagliptin and nataglinide.

Preferably said agents with a promoter of the insulin secretion are GLP-1 agonists, more preferably said agents are liraglutide or exenatide. Preferably said agents with dopamine receptor 2 agonism are cabergoline and bromocriptine.

Preferably, said chemotherapeutic agent is temozolomide.

According to a further preferred embodiment, the compound of formula (I) or the pharmaceutically acceptable salts and/or solvates thereof is administered together with at least one active principle simultaneously, separately or sequentially.

The invention will be further illustrated in greater details in the following experimental section. The following examples are not intended to limit the invention.

### EXPERIMENTAL SECTION

### ITF2984 Structure

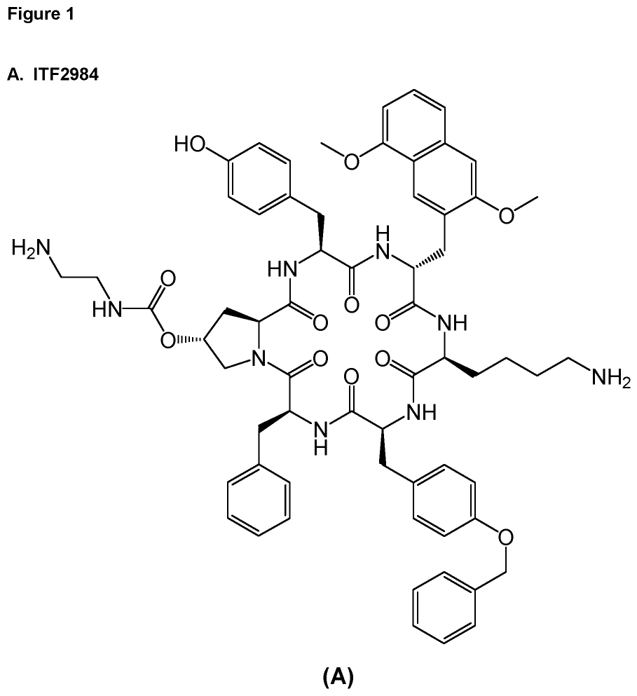

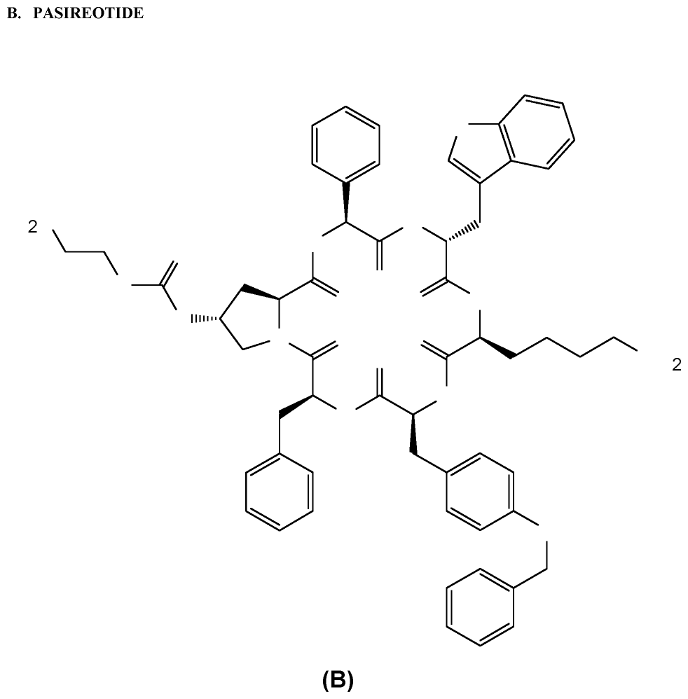

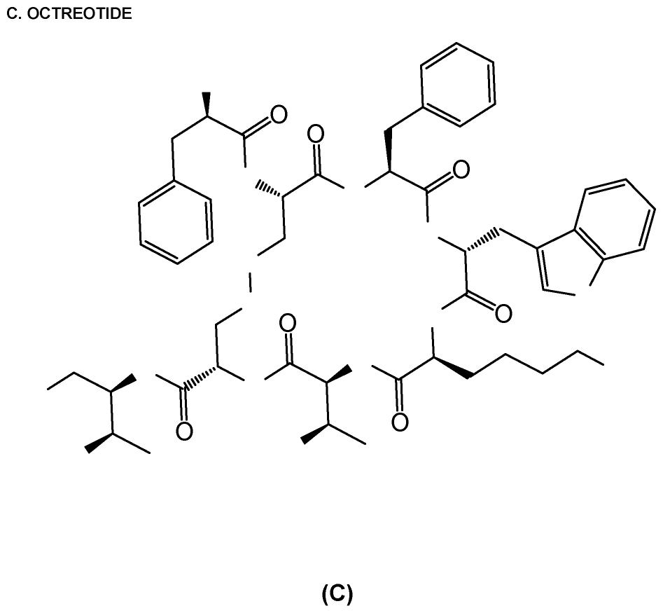

ITF2984 (formula reported in FIG. 1, panel A), a novel SST pan-agonist cyclic hexapeptide, was discovered in a medicinal chemistry program aimed at identifying pan-agonists with improved properties versus first-generation SSAs. The compound showed high binding affinity for SSTR1, 2, 3 and 5, with respect to other known somatostatin analogs such as Pasireotide and Octreotide, with IC₅₀ values in the nanomolar range for all receptors. IC₅₀ values are from one representative experiment. Confidence interval from 3 independent experiments (Table 1).

When compared to octreotide, ITF2984 showed higher affinity for SSTR1, SSTR3 and SSTR5 and lower affinity for SSTR2, whereas, relative to Pasireotide, it exhibits higher affinity for human SSTR1, SSTR2 and SSTR3 (Table 1).

Table 1 shows a profile of ITF2984 activities compared to Pasireotide and Octreotide

We notice that particularly the IC₅₀ values of ITF2984 on SSTR3 were about 1 order of magnitude lower than those obtained with either Octreotide or with Pasireotide.

A molecular modeling approach was used to rationalize the structural basis for the higher affinity of ITF2984 for SSTR3. To this end, the SSTRs structures from GPCRdb²⁸, an open access repository of G-coupled protein receptor structures, were used. Models of receptors SSTR1-4 are mainly based on the κ-opioid receptor (or KOP, sequence similarity 60-66%, pdb codes 6B73 and 6VI4 for active and inactive conformer, respectively), whereas SSTR5 is more similar to the 8-opioid receptor. The relevance of using the opioid receptors in this modeling exercise was experimentally confirmed by ITF2984 and Pasireotide single dose binding assays on this particular GPCR family where both hexapeptides are able to fully replace agonists at 10⁻⁵ M (Table 2).

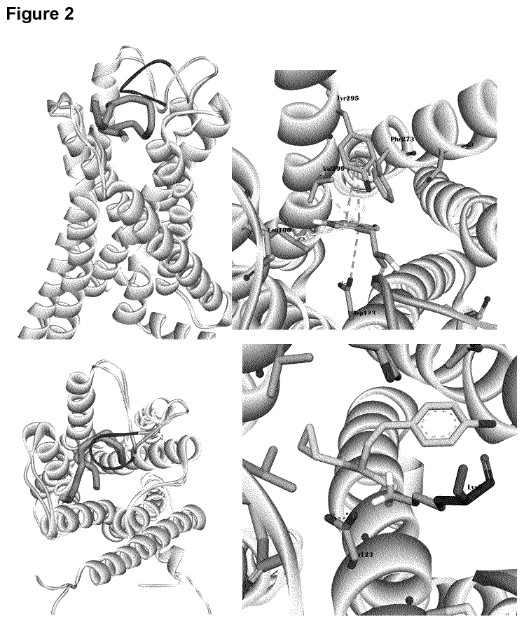

To explain the different behavior of ITF2984 and Pasireotide on SSTR2 and SSTR3, we initially explored the somatostatin-receptor interaction using a SST14 conformer derived from an NMR study of SST14 in 5% D-mannitol solution (first conformer among ten geometries, PDB code 2MI1). SST14 docks well into SSTR3 and the highest ZDOCK score pose interacts with transmembrane helices TM5, TM6 and TM7 and with Phe7 apparently close to an extracellular loop linking TM4 and TM5 (EL4-5). Unfortunately, an equivalent docking calculation could not be performed for SRIF-14 and SSTR2, due to the longer extracellular loop between TM4 an TM5: this loop conformation obstructs the entrance to an internal space delimited by the transmembrane helices in SSTR2 (FIG. 2).

The SST14 binding geometry in the SSTR3 model has a series of interesting features:
i) Lys9 is anchored to Asp123 and the ζ Nitrogen atom occupies same region of NH₄⁺group of the KOP agonist MP1104 when the active form of K-opioid (PDB code 6B73) is superposed to SSTR3-SST14 best docking pose complex,
ii) Trp8 fits a hydrophobic pocket delimited by Leu100, Val299, Tyr295and Phe273, buried in a local non-covalent bonds network,
iii) The ligand residues Phe7-Trp8-Lys9-Thr10 generate a distorted β-II′ turn.

The distorted β-II′ turn found in the most probable SRIF-14 binding conformer suggests that peptide agonists showing higher stability of such secondary structure motif could be preferred. An NMR study²⁹ on Pasireotide and L-363,301 revealed a higher flexibility of the former, relative to the other cyclic hexapeptide, and the larger degree of freedom was considered to be the reason of Pasireotide's ability to fit better in all receptor binding sites.

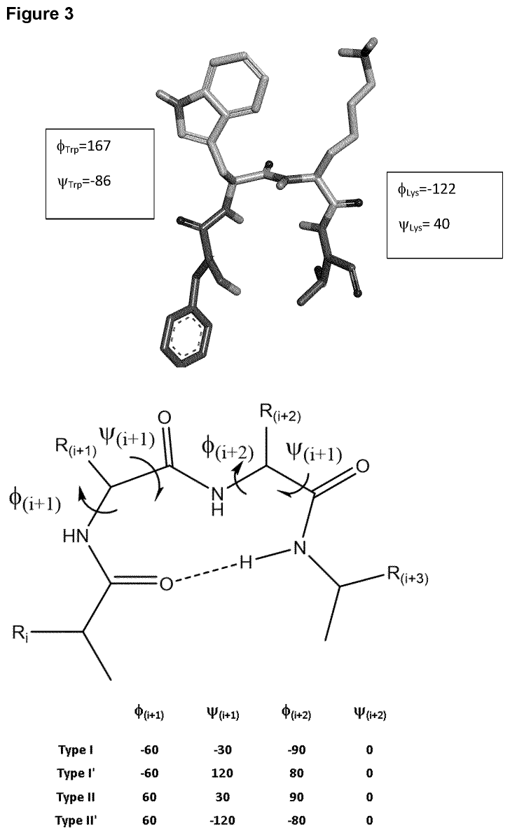

Analogous experiments on ITF2984 (FIG. 3) show a higher β-II′ turn probability for ITF2984 relative to Pasireotide, due to the larger number of molecular dynamics snapshots detected in trajectory analysis (30% instead of 12%). The β-II′-turn probability is even higher (58%) in Compound ITF2842. Comparison of binding results on SSTR1-3 and SSTR5 for all three molecules (Table3) shows a correlation between stability of the β-turn and potency (and selectivity) vs SSTR3. The higher the stability of the B-turn, the higher is the selectivity for SSTR3.

We conclude that there is a plausible structural hypothesis to explain the increased affinity of ITF2984 for SSTR3.

TABLE 3
Comparison between β-turn II′ probability
and binding ratio SSTR-2 vs SSTR-3.

| Compound | β-II′ turn probability | SSTR2/SSTR3 |
|---|---|---|
| Pasireotide | 12% | 2, 12 |
| ITF2984 | 30% | 4, 76 |
| ITF2842 | 58% | 21, 91 |

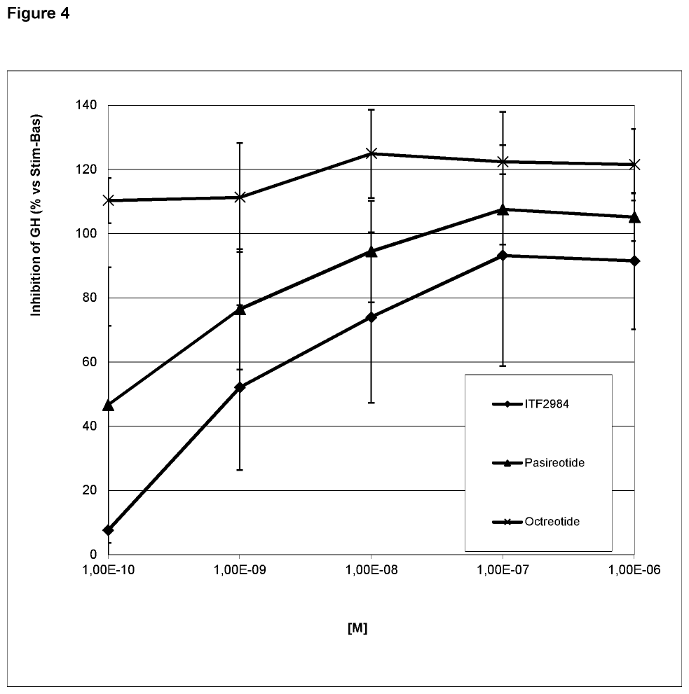

We further characterized the biological activity of ITF2984: this molecule potently inhibited GH release from rat anterior pituitary primary cell cultures with no statistical difference with respect to Pasireotide (FIG. 4). ITF2984 further inhibited pentobarbital-induced GH release in rats and decreased IGF1 levels in rats and dogs.

To highlight mechanistic differences between ITF2984 and other SSTR agonists, we investigated SSTR internalization using two different experimental models:

HEK293 cells transfected with human SSTRs, and U20S cells transfected with human GFP-tagged receptors (SSTR2-tGFP, SSTR3-tGFP, SSTR5-tGFP). SST14 and SST28 were included as reference compounds in the first and second experiments, respectively.

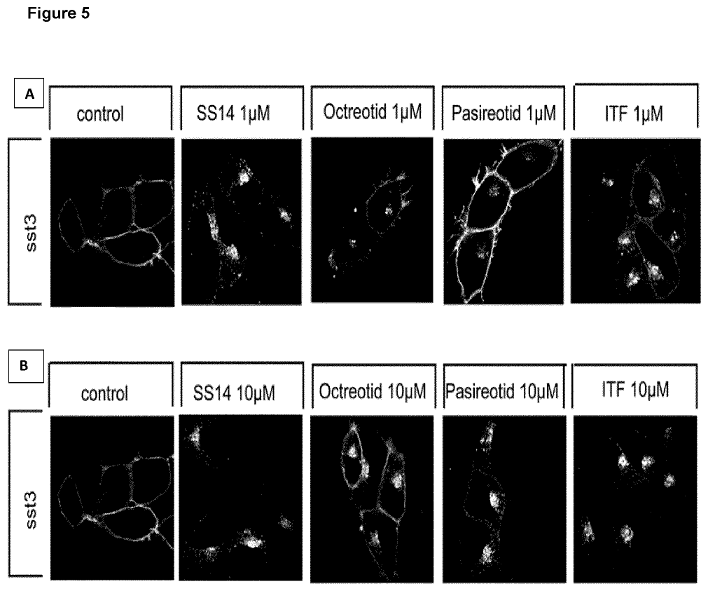

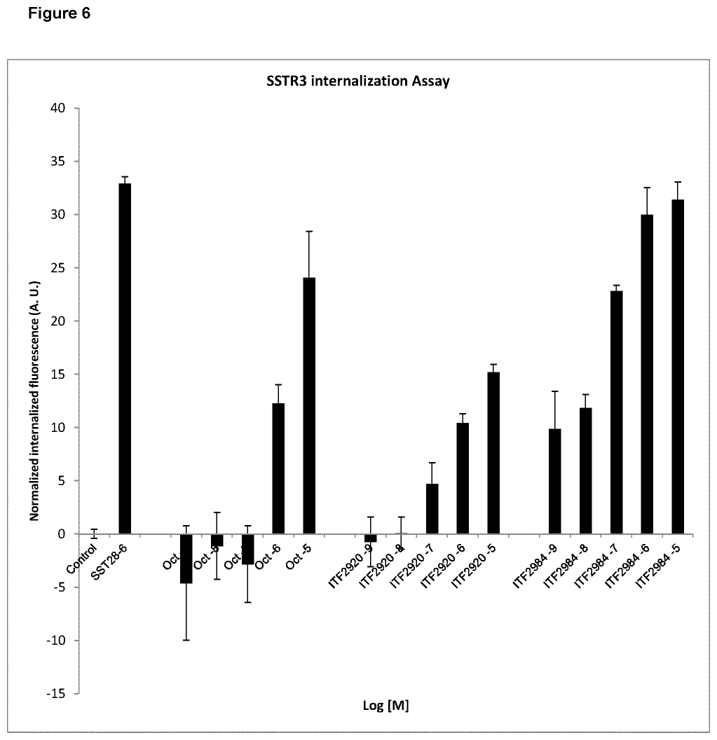

In transfected HEK293 cells, ITF2984 induced a strong internalization of SSTR3 which was comparable to that induced by SST14 and significantly higher than that induced by Octreotide or Pasireotide (FIG. 5A, 5B). In contrast, only a partial internalization of SSTR2 and SSTR5 was observed. In U2OS cells, all compounds increased SSTR2-tGFP internalization in a dose-dependent manner when compared to untreated control, with Octreotide being the most potent internalization inducer, followed by Pasireotide and ITF2984. In SSTR5-tGFP-transfected cells, the increase in receptor internalization due to the treatment with SSAs was similar to the effect of SST albeit lower (about fifty percent of SST28) (Table 4, FIG. 6).

TABLE 4
Internalization of SSTR2, 3, 5 following treatments with SST28,
Pasireotide, ITF2984 and Octreotide in U2OS transfected cells
(Mean of internalized fluorescence in Arbitrary Units (AU)).

| | | SST28 | Octreotide | Pasireotide | ITF2984 |
|---|---|---|---|---|---|
| | Control | 1 μM | 1 μM-10 μM | 1 μM-10 μM | 1 μM-10 μM |
| SSTR2 | 27.6 | 51.4 | 74.1-81.0 | 49.2-50.5 | 37.3-35.9 |
| SSTR3 | 31.1 | 64.0 | 43.4-55.2 | 41.6-46.3 | 61.1-62.5 |
| SSTR5 | 29.0 | 84.5 | 34.6-39.3 | 40.5-43.9 | 34.6-42.0 |

Lastly, in SSTR3-tGFP-transfected cells, ITF2984 was the most potent inducer of receptor internalization showing an increment comparable to SST28 at 1 μM. At the same concentration, the effect of Octreotide and Pasireotide was significantly less pronounced.

We conclude that ITF2984 induces SSTR3 internalization more effectively than Pasireotide or Octreotide in two different cell lines.

### Evaluation of SSA-Induced Activation of Human SSTR2, SSTR3 and SSTR5 Receptors Using Phosphosite-Specific Antibodies

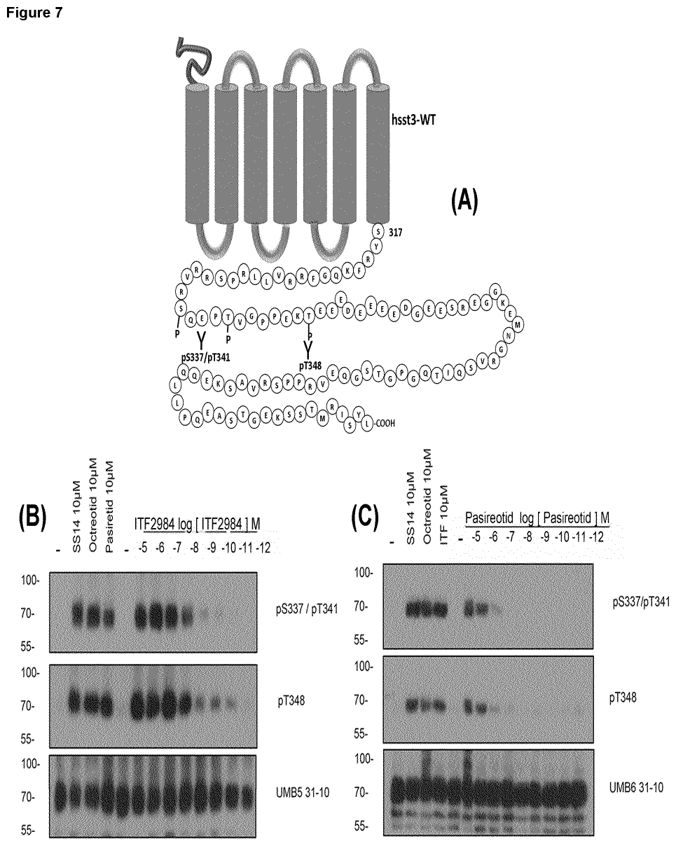

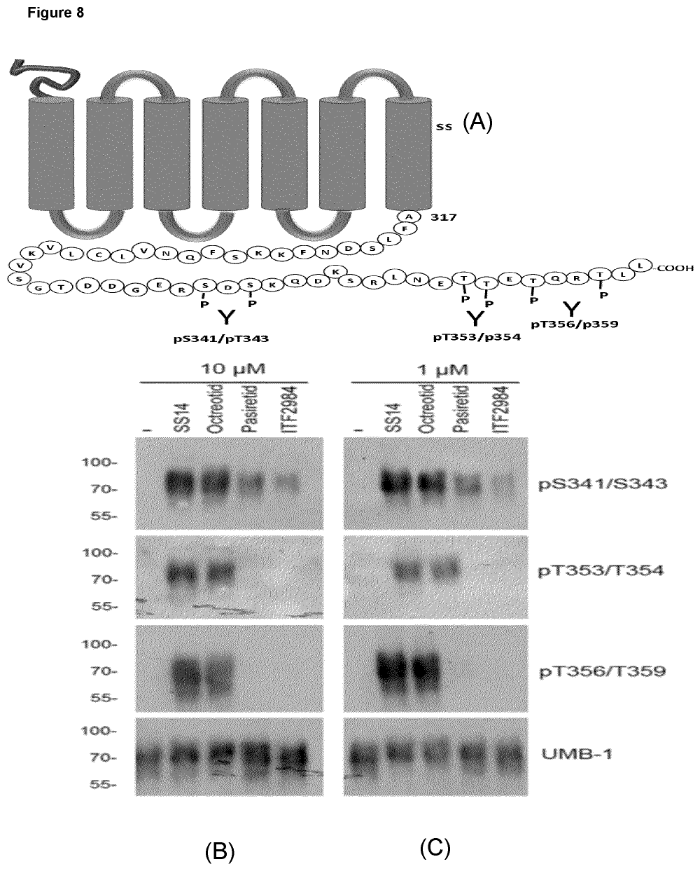

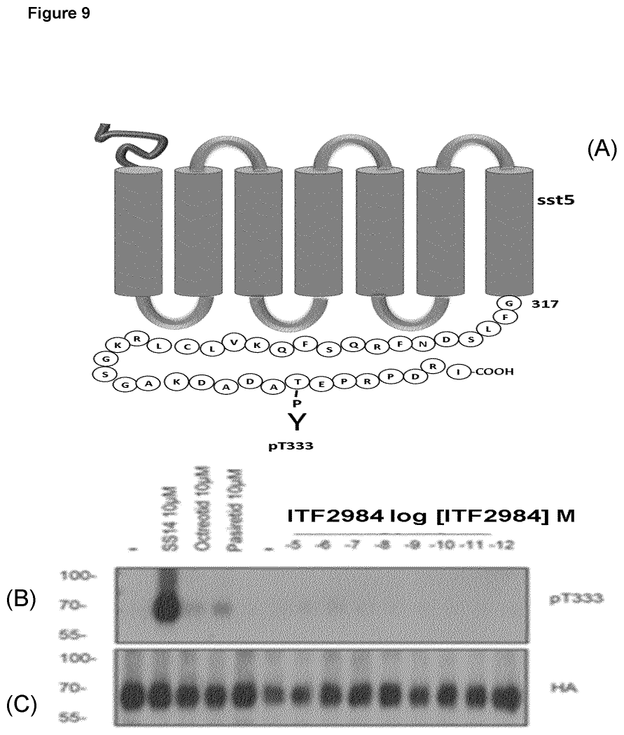

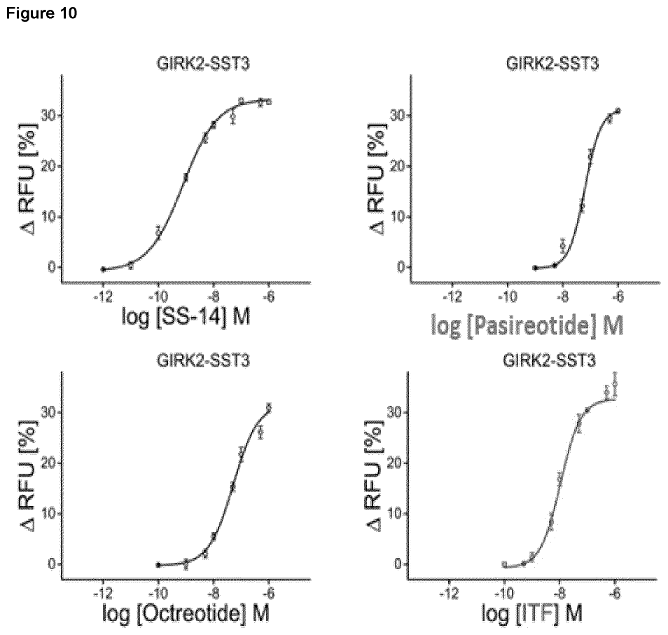

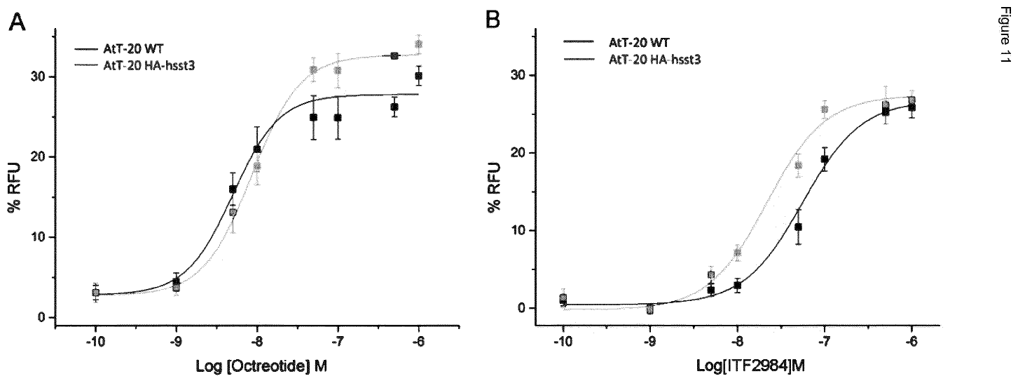

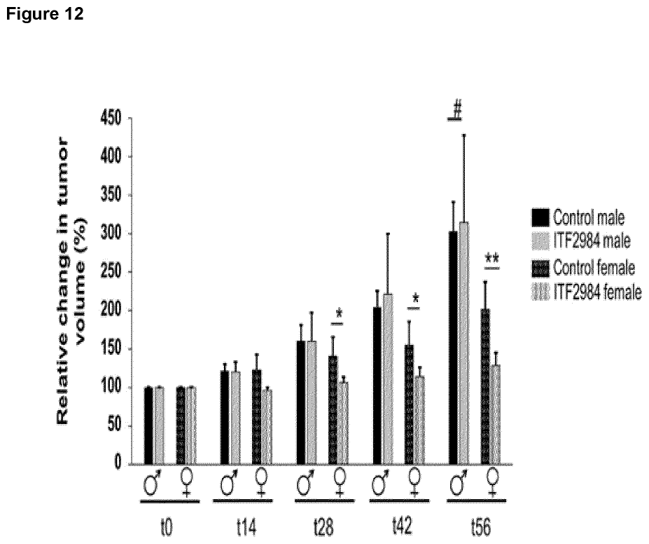

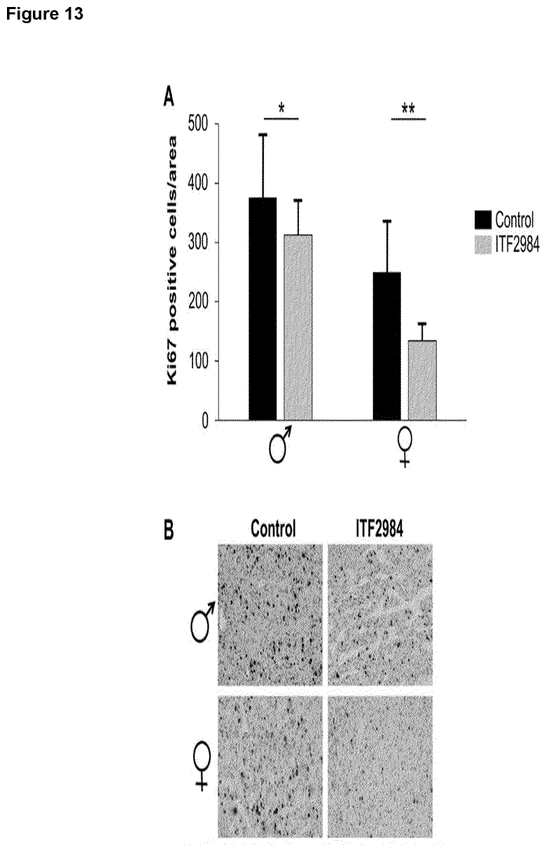

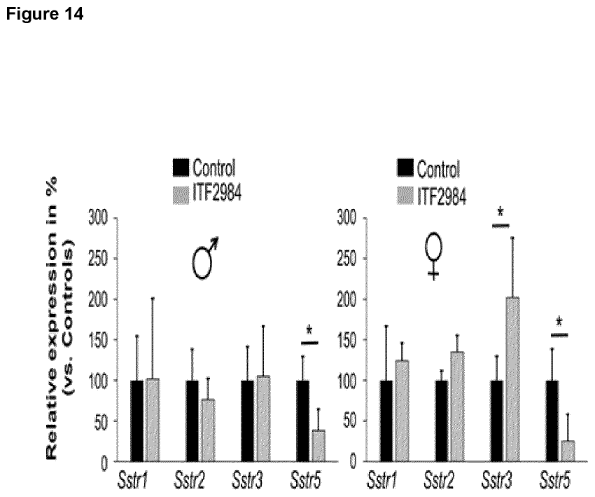

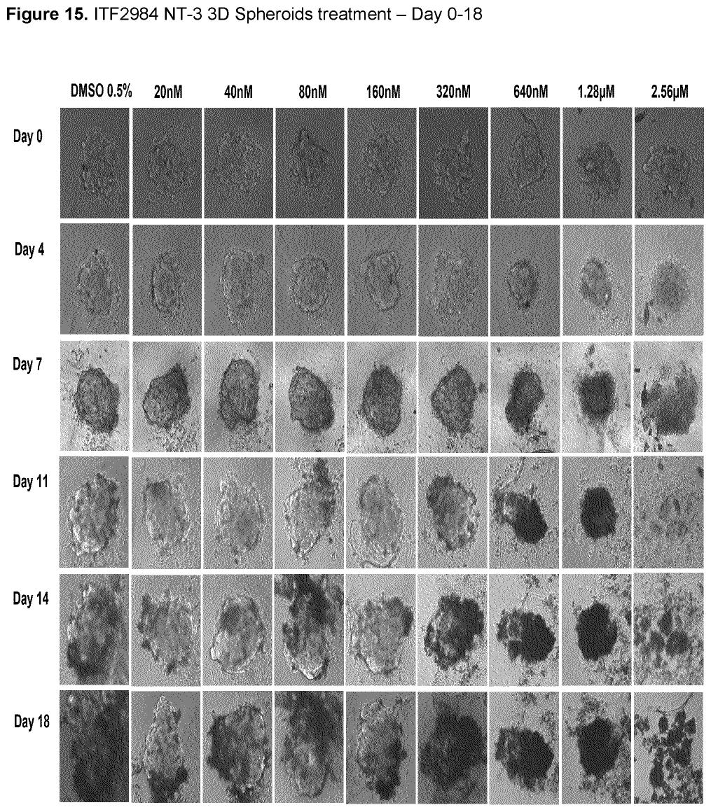

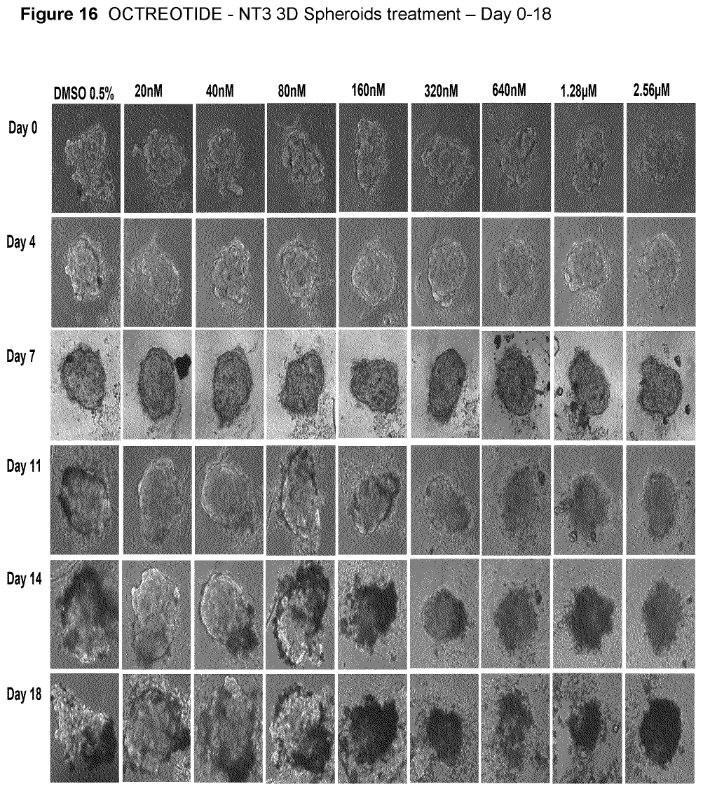

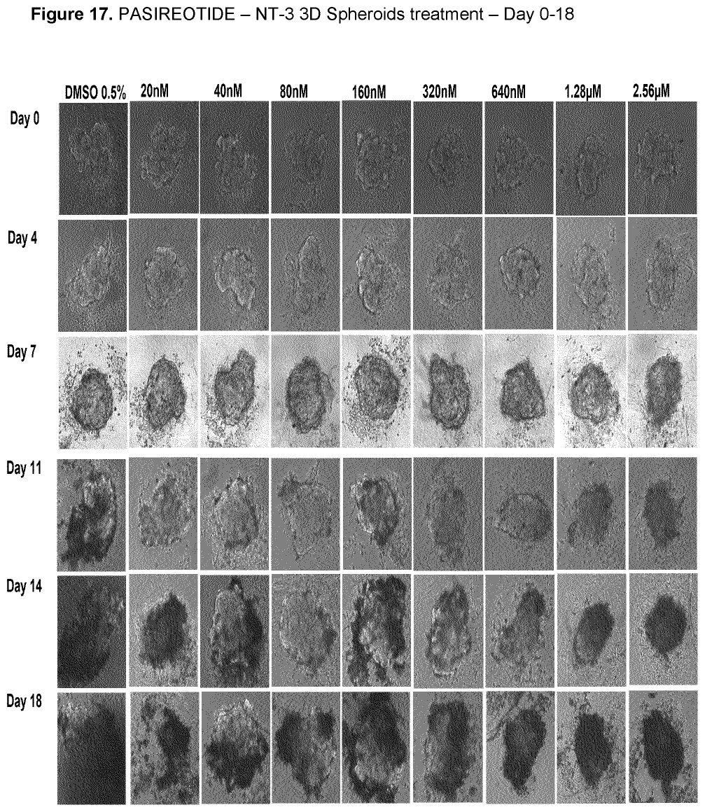

Next, the activation of human SSTRs induced by SSAs was tested by western blot using phosphosite-specific antibodies directed against the following residues: for SSTR2: S341/pS343, T353/T354, T356/T359; for SSTR3: S337/T341, T348; for SSTR5: T333.30 ITF2984 induced the full phosphorylation of SSTR3 in a dose dependent manner (FIG. 7B, C) and in a pharmacologically relevant, nanomolar dose range, whereas Pasireotide only induced a weak effect in a supra-pharmacologic, micromolar, dose-range. In SSTR2, both cyclo-hexapeptides induced a selective phosphorylation of S341/S343, whereas SST14 and Octreotide lead to the full phosphorylation of SSTR2 at all investigated sites (FIG. 8B, C). Finally, the SSTR5 pT333 phosphosite was only partially affected by all tested compounds, with Pasireotide showing the most pronounced effect (FIG. 9B, C).

Agonist-mediated G protein-signaling of SSTR3 in HEK293 cells and in AtT20 SSTR3 transfected cells.

Somatostatin receptor signaling is known to activate G protein coupled inwardly rectifying potassium (GIRK) channels.³⁰

Therefore, G protein-signaling mediated by SSTR agonists was studied using a GIRK-based fluorescence membrane potential assay both in HEK293 cells transfected with hSSTR3, and in AtT20 wild-type as well as hSSTR3-transfected cells.

Stimulation of HEK293 cells stably expressing human SSTR3 receptor with SST14, Octreotide, Pasireotide and ITF2984 resulted in a dose-dependent reduction in the fluorescent signal of the FMP dye, with ITF2984 being more potent than Octreotide or Pasireotide (FIG. 10). To better characterize ITF2984, the GIRK channel activation was also applied to SSTR2 and SSTR5. In HEK293-GIRK2-GFP-HA-hSST2 the most potent agonist was Octreotide followed by Pasireotide and ITF2984, while in HEK293-GIRK2-GFP-HA-hSST5 the highest activation was induced by Pasireotide. Results of agonist-mediated G protein-signalling of SSTR2, SSTR3, SSTR5 in HEK293 cells are summarized in Table 5.

TABLE 5
SST receptor activation by SST14, Octreotide, Pasireotide and ITF2984
in HEK293-GIRK2-GFP-HA-hSST2, hSST3 or hSST5 transfected cells.
Results are expressed as Mean ± SEM, calculated from duplicate
determinations from three independent experiments.

| | Compound activity (EC₅₀ nM) | | | |
|---|---|---|---|---|
| SSTR | SST-14 | Octreotide | Pasireotide | ITF2984 |
| SSTR2 | 0.4 ± 0.03 | 4.0 ± 0.5 | 60.0 ± 16.3 | 311.6 ± 29.3 |
| SSTR3 | 0.8 ± 0.1 | 51.9 ± 10.0 | 63.0 ± 7.5 | 10.7 ± 1.7 |
| SSTR5 | 0.5 ± 0.1 | 54.4 ± 6.6 | 16.5 ± 2.5 | 95.1 ± 15.2 |

Additionally, G protein-signaling mediated by Octreotide and ITF2984 was analyzed in mouse corticotroph tumor AtT-20 cells, which endogenously express GIRK1/2 channels as well as SSTR2 and SSTR5 receptors. Exogenous expression of SSTR3 receptor resulted in a leftward shift of the ITF2984-mediated dose-response curve, but not of the dose response curve obtained with octreotide, suggesting engagement of SSTR3 by ITF2984, but not by Octreotide under these conditions (FIG. 11). These data agree with those obtained with hSSTR3 transfected HEK293 cell line.

### Efficacy of ITF2984 Against Endogenous NFPAs In Vivo

Encouraged by the in vitro findings, we decided to study the in vivo anti-tumor activity ITF2984 in the MENX (homozygous mutant) rat model, which is the only spontaneous, endogenous model where NFPAs that closely resemble their human counterpart develop with complete penetrance.^{16,17} NFPAs that develop in this model were found to recapitulate also the SSTR expression pattern of their human counterparts, showing high SSTR3 expression. Interestingly, SSTR3 levels in MEX rat pituitary tumors were found to be gender-specific, with higher expression observed in females. This pattern may extend also to human NFPAs. Rats of both genders were treated for 56 days with ITF2984 or placebo, and tumor growth was monitored longitudinally using high-resolution magnetic resonance imaging (MRI).

Male rats treated with ITF2984 and control group members showed a rapid increase of relative tumor volume, (FIG. 12) exhibiting logarithmic value best fitted by a Linear Mixed Effect model (LME) with quadratic time effects In contrast, female rats treated with ITF2984 showed only a slight increase in tumor volume during treatment .indicating a linear growth instead. The difference in time slopes between sexes for the ITF2984-treated rats was significant (p-value=0.0251). This indicates that female mutant rats responded significantly better to ITF2984 when compared to the males. Remarkably, although two female rats had already relatively large tumors at the beginning of the study, ITF2984 kept tumor growth under control and these two animals showed no signs of discomfort at the end of treatment. When combining both sexes, the overall reduction in tumor growth in ITF2984-treated rats (used as proxy for drug response) versus that in control rats, as assessed by the best fitted LME, was modest. However, when sexes were analyzed separately, drug-treated female rats showed a suppression of tumor growth when compared to their placebo-treated counterpart, indicating that the former group responded to ITF2984.

### Effect of ITF2984 on NFPAs Proliferation Rates

At the end of the treatment, pituitary tissues were collected for ex vivo analyses. Staining for Ki67 was performed on all tumors of rats treated with either placebo or ITF2984, and the number of Ki67-positive cells per 100.000 μm² was counted. Placebo-treated (control) NFPAs showed an average of 375 (males) and 312 (females) Ki67-positive cells per area, as reported (FIG. 13). Not surprisingly, in the control group there was a positive trend between the number of Ki67-positive cells and absolute tumor volume in males and females. In tumors of rats treated with ITF2984, the number of Ki-67-positive cells dropped to an average of 250 (−33.5%) for male and to 134 ('157%) for female rats (FIG. 13). The difference in the number of Ki67-positive cells between placebo-and ITF2984-treated female rats is significant (p=0.031). Changes in tumor cell proliferation correlate with the changes in tumor volume determined by MRI: ITF2984 suppressed tumor growth more effectively in females then males, and this went together with a stronger reduction in NFPA cells proliferation in the former.

### Expression of SSTRs in Rat NFPAs

The expression level of the various Sstr genes was assessed in rat tumors at the end of treatment by measuring the copy number for each transcript (absolute quantification) via quantitative RT-PCR. Tumors from the 2 animal groups were also stained with antibodies against SSTR1, 2, 3 and 5 using immunohistochemistry (IHC) and the results confirmed the high expression of SSTR3 in female rats³¹. ITF2984 administration led to a downregulation of Sstr5 expression in both sexes, and to an increase in Sstr3 mRNAs, but only in females (FIG. 14). This higher expression of SSTR3 in female rats explain the greater effect of ITF2984 in female sex. It has not been demonstrated a statistically significantly higher expression of SSTR3 also in human female.

### Activity of ITF2984 Compared to Octreotide and Pasireotide on NT-3 Cell Line Spheroids

### Method-NT-3 Cell Line Spheroids Generation and Treatment

NT-3 well-differentiated pancreatic neuroendocrine (PanNET) cell line (32) is an in-vitro model for pancreatic tumours.

Said cells were cultured in RPMI 1640 Medium GlutaMAX™ with 10% FBS, 1% Penicillin/Streptomycin, 20ng/ml EGF (AF-100-15, Peprotech), 10 ng/ml FGF (100-18B, Peprotech).

3D spheroids were generated by seeding 2000 cells/well in a 96-well ULA plate (Corning). Four replicates for each condition were seeded.

The spheroid formation required 4 days before starting the treatment. Stock solutions of ITF2984, Pasireotide and Octreotide have been prepared in 100% DMSO and working solutions in complete growth medium containing 0.5% DMSO at 2560, 1280, 640, 320, 160, 80, 40, 20 nM.

The treatment was repeated every 3 days by replacing 50% of the medium (to avoid spheroid removal) with new medium containing 2× desired drug concentration.

Spheroid morphology was monitored every day during the treatment and pictures were taken on day 0, 4, 7, 11, 14, 18. Day 0 corresponds to the first day of treatment. Pictures were taken from the same well every time point.

Results-ITF2984 cytotoxic activity on NT-3 spheroids

The recently developed NT-3 cell line is a well differentiated PanNET expressing high levels of SSTRs, especially SSTR3 (32). ITF2984 has been tested in dose-response for its cytotoxic activity on NT-3 spheroids in parallel to Octreotide and Pasireotide. Spheroid growth/survival was followed for eighteen days.

As shown in FIG. 15 ITF2984 reduced the spheroid volume starting from day 7 at concentration ≥640 nM. At 2.56 μM spheroids are completed destroyed. The same phenomenon is less evident in Octreotide and Pasireotide-treated spheroids, which still maintain their morphology at the highest concentration (FIGS. 16 and 17).

Said results can therefore demonstrate that ITF2984 can be used as a more effective drug for the treatment of pancreatic cancer, considering that the cytotoxic activity observed in NT-3 spheroids corresponds to anti-tumor activity.

### REFERENCES

(1) Ntali, G.; Wass, J. A. Epidemiology, Clinical Presentation and Diagnosis of Non-Functioning Pituitary Adenomas. Pituitary 2018, 21 (2), 111-118, DOI: 10.1007/s11102-018-0869-3.
(2) Drummond, J.; Roncaroli, F.; Grossman, A. B.; Korbonits, M. Clinical and Pathological Aspects of Silent Pituitary Adenomas. J. Clin. Endocrinol. Metab. 2019, 104 (7), 2473-2489, DOI: 10.1210/jc.2018-00688.
(3) Lee, M.; Lupp, A.; Mendoza, N.; Martin, N.; Beschorner, R.; Honegger, J.; Schlegel, J.; Shively, T.; Pulz, E.; Schulz, S.; Roncaroli, F.; Pellegata, N. S. SSTR3 Is a Putative Target for the Medical Treatment of Gonadotroph Adenomas of the Pituitary. Endocr. Relat. Cancer 2015, 22 (1), 111-119, DOI: 10.1530/ERC-14-0472.
(4) De Divitiis, E.; Laws, E. R.; Giani, U.; Iuliano, S. L.; De Divitiis, O.; Apuzzo, M. L. J. The Current Status of Endoscopy in Transsphenoidal Surgery: An International Survey. World Neurosurg. 2015, 83 (4), 447-454,DOI: 10.1016/j.wneu.2014.12.029.
(5) Reddy, R.; Cudlip, S.; Byrne, J. V.; Karavitaki, N.; Wass, J. A. H. Can We Ever Stop Imaging in Surgically Treated and Radiotherapy-Naive Patients with Non-Functioning Pituitary Adenoma? Eur. J. Endocrinol. 2011, 165 (5), 739-744, DOI: 10.1530/EJE-11-0566.
(6) Tampourlou, M.; Ntali, G.; Ahmed, S.; Arlt, W.; Ayuk, J.; Byrne, J. V.; Chavda, S.; Cudlip, S.; Gittoes, N.; Grossman, A.; Mitchell, R.; O'Reilly, M. W.; Paluzzi, A.; Toogood, A.; Wass, J. A. H.; Karavitaki, N. Outcome of Nonfunctioning Pituitary Adenomas That Regrow after Primary Treatment: A Study from Two Large UK Centers. J. Clin. Endocrinol. Metab. 2017, 102 (6), 1889-1897, DOI: 10.1210/jc.2016-4061.
(7) Vieira Neto, L.; Boguszewski, C. L.; de Araújo, L. A.; Bronstein, M. D.; Miranda, P. A. C.; Musolino, N. R. d. C.; Naves, L. A.; Vilar, L.; Ribeiro-Oliveira Júnior, A.; Gadelha, M. R. A Review on the Diagnosis and Treatment of Patients with Clinically Nonfunctioning Pituitary Adenoma by the Neuroendocrinology Department of the Brazilian Society of Endocrinology and Metabolism. Arch. Endocrinol. Metab. 2016, 60 (4), 374-390, DOI: 10.1590/2359-3997000000179.
(8) Ilie, M. D.; Raverot, G. Treatment Options for Gonadotroph Tumors: Current State and Perspectives. J. Clin. Endocrinol. Metab. 2020, 105 (10), 3507-3518, DOI: 10.1210/clinem/dgaa497.
(9) Keane, F.; Egan, A. M.; Navin, P.; Brett, F.; Dennedy, M. C. Gonadotropin-Releasing Hormone Agonist-Induced Pituitary Apoplexy. Endocrinol. Diabetes Metab. Case Reports 2016, 2016 (June), DOI: 10.1530/edm-16-0021.
(10) Theodoropoulou, M.; Stalla, G. K. Somatostatin Receptors: From Signaling to Clinical Practice. Front. Neuroendocrinol. 2013, 34 (3), 228-252,DOI: 10.1016/j.yfrne.2013.07.005.
(11) Ben-Shlomo, A.; Melmed, S. Somatostatin Agonists for Treatment of Acromegaly. Mol. Cell. Endocrinol. 2008, 286 (1-2), 192-198, DOI: 10.1016/j.mce.2007.11.024.
(12) Grozinsky-Glasberg, S.; Shimon, I.; Korbonits, M.; Grossman, A. B. Somatostatin Analogues in the Control of Neuroendocrine Tumours: Efficacy and Mechanisms. Endocr. Relat. Cancer 2008, 15 (3), 701-720,DOI: 10.1677/ERC-07-0288.
(13) Petersenn, S. Medical Therapy of Aggressive Pituitary Tumors. Exp. Clin. Endocrinol. Diabetes 2021, 129 (3), 186-193, DOI: 10.1055/a-1331-6939.
(14) Gomes-Porras, M.; Cárdenas-Salas, J.; Álvarez-Escolá, C. Somatostatin Analogs in Clinical Practice: A Review. Int. J. Mol. Sci. 2020, 21 (5), 1-27, DOI: 10.3390/ijms21051682.
(15) Stahl, P. H. Handbook of Pharmaceutical Salts Properties, Selection, and Use; John Wiley & Sons, 2008.
(16) Marinoni, I.; Lee, M.; Mountford, S.; Perren, A.; Bravi, I.; Jennen, L.; Feuchtinger, A.; Drouin, J.; Roncaroli, F.; Pellegata, N. S. Characterization of MENX-Associated Pituitary Tumours. Neuropathol. Appl. Neurobiol. 2013, 39 (3), 256-269, DOI: 10.1111/j. 1365-2990.2012.01278.x.
(17) Lee, M.; Marinoni, I.; Irmler, M.; Psaras, T.; Honegger, J. B.; Beschorner, R.; Anastasov, N.; Beckers, J.; Theodoropoulou, M.; Roncaroli, F.; Pellegata, N. S. Transcriptome Analysis of MENX-Associated Rat Pituitary Adenomas Identifies Novel Molecular Mechanisms Involved in the Pathogenesis of Human Pituitary Gonadotroph Adenomas. Acta Neuropathol. 2013, 126 (1), 137-150, DOI: 10.1007/s00401-013-1132-7.
(18) Oystese, K. A.; Casar-Borota, O.; Normann, K. R.; Zucknick, M.; Berg, J. P.; Bollerslev, J. Estrogen Receptor a, a Sex-Dependent Predictor of Aggressiveness in Nonfunctioning Pituitary Adenomas: Sstr and Sex Hormone Receptor Distribution in NFPA. J. Clin. Endocrinol. Metab. 2017, 102 (9), 3581-3590, DOI: 10.1210/jc.2017-00792.
(19) Diana llie, M.; Vasiljevic, A.; Louvet, C.; Jouanneau, E.; Raverot, G. Gonadotroph Tumors Show Subtype Differences That Might Have Implications for Therapy. Cancers (Basel). 2020, 12 (4), 1-12, DOI: 10.3390/cancers12041012.
(20) Shahbaz, M.; Ruliang, F.; Xu, Z.; Benjia, L.; Cong, W.; Zhaobin, H.; Jun, N. MRNA Expression of Somatostatin Receptor Subtypes SSTR-2, SSTR-3, and SSTR-5 and Its Significance in Pancreatic Cancer. World J. Surg. Oncol. 2015, 13 (1), 1-6, DOI: 10.1186/s12957-015-0467-z.
(21) Elston, M. S.; Meyer-Rochow, G. Y.; Conaglen, H. M.; Clarkson, A.; Clifton-Bligh, R. J.; Conaglen, J. V.; Gill, A. J. Increased SSTR2A and SSTR3 Expression in Succinate Dehydrogenase-Deficient Pheochromocytomas and Paragangliomas. Hum. Pathol. 2015, 46 (3), 390-396, DOI: 10.1016/j.humpath.2014.11.012.
(22) Kanakis, G.; Grimelius, L.; Spathis, A.; Tringidou, R.; Rassidakis, G. Z.; Öberg, K.; Kaltsas, G.; Tsolakis, A. V. Expression of Somatostatin Receptors 1-5 and Dopamine Receptor 2 in Lung Carcinoids: Implications for a Therapeutic Role. Neuroendocrinology 2015, 101 (3), 211-222, DOI: 10.1159/000381061.
(23) Frati, A.; Rouzier, R.; Lesieur, B.; Werkoff, G.; Antoine, M.; Rodenas, A.; Darai, E.; Chereau, E. Expression of Somatostatin Type-2 and-4Receptor and Correlation with Histological Type in Breast Cancer. Anticancer Res. 2014, 34 (8), 3997-4004.
(24) Florio, T.; Morini, M.; Villa, V.; Arena, S.; Corsaro, A.; Thellung, S.; Culler, M. D.; Pfeffer, U.; Noonan, D. M.; Schettini, G.; Albini, A. Somatostatin Inhibits Tumor Angiogenesis and Growth via Somatostatin Receptor-3-Mediated Regulation of Endothelial Nitric Oxide Synthase and Mitogen-Activated Protein Kinase Activities. Endocrinology 2003, 144 (4), 1574-1584, DOI: 10.1210/en.2002-220949.
(25) Zatelli, M. C.; Piccin, D.; Vignali, C.; Tagliati, F.; Ambrosio, M. R.; Bondanelli, M.; Cimino, V.; Bianchi, A.; Schmid, H. A.; Scanarini, M.; Pontecorvi, A.; De Marinis, L.; Maira, G.; Degli Uberti, E. C. Pasireotide, a Multiple Somatostatin Receptor Subtypes Ligand, Reduces Cell Viability in Non-Functioning Pituitary Adenomas by Inhibiting Vascular Endothelial Growth Factor Secretion. Endocr. Relat. Cancer 2007, 14 (1), 91-102, DOI: 10.1677/ERC-06-0026.
(26) Günther, T.; Tulipano, G.; Dournaud, P.; Bousquet, C.; Csaba, Z.; Kreienkamp, H. J.; Lupp, A.; Korbonits, M.; Castaño, J. P.; Wester, H. J.; Culler, M.; Melmed, S.; Schulz, S. International Union of Basic and Clinical Pharmacology. CV. Somatostatin Receptors: Structure, Function, Ligands, and New Nomenclature. Pharmacol. Rev. 2018, 70 (4), 763-835, DOI: 10.1124/pr.117.015388.
(27) Gatto, F.; Feelders, R. A.; Van Der Pas, R.; Kros, J. M.; Waaijers, M.; Sprij-Mooij, D.; Neggers, S. J. C. M. M.; Van Der Lelij, A. J.; Minuto, F.; Lamberts, S. W. J.; De Herder, W. W.; Ferone, D.; Hofland, L. J. Immunoreactivity Score Using an Anti-Sst2A Receptor Monoclonal Antibody Strongly Predicts the Biochemical Response to Adjuvant Treatment with Somatostatin Analogs in Acromegaly. J. Clin. Endocrinol. Metab. 2013, 98 (1), E66-E71, DOI: 10.1210/jc.2012-2609.
(28) Kooistra, A. J.; Mordalski, S.; Esguerra, M.; Mamyrbekov, A.; Munk, C.; Keser, M.; Gloriam, D. E. OUP Accepted Manuscript. Nucleic Acids Res. 2020, 49 (December 2020), 335-343, DOI: 10.1093/nar/gkaa1080.
(29) A. Stevenazzi, G. Sandrone, M. Pinori, P. M. NMR and Molecular Dynamics Analysis of SOM230: A Pluripotent Somatostatin Analogue 9th INTERNATIONAL SYMPOSIUM ON SOLID PHASE SYNTHESIS, COMPLEMENTARY SOLUTION METHODS & COMBINATORIAL LIBRARIES 2nd INTERNATIONAL SYMPOSIUM ON PROTEOMIC, COMBINATORIAL & OTH; 2007.
(30) Lehmann, A.; Kliewer, A.; Günther, T.; Nagel, F.; Schulz, S. Identification of Phosphorylation Sites Regulating Sst3 Somatostatin Receptor Trafficking. Mol. Endocrinol. 2016, 30 (6), 645-659, DOI: 10.1210/me.2015-1244.
(31) Gulde, S.; Wiedemann, T.; Schillmaier, M.; Valença, I.; Lupp, A.; Steiger, K.; Yen, H. Y.; Bäuerle, S.; Notni, J.; Luque, R.; Schmid, H.; Schulz, S.; Ankerst, D. P.; Schilling, F.; Pellegata, N. S. Gender-Specific Efficacy Revealed by Head-to-Head Comparison of Pasireotide and Octreotide in a Representative in Vivo Model of Nonfunctioning Pituitary Tumors. Cancers (Basel). 2021, 13 (12), DOI: 10.3390/cancers13123097.
(32) Daniel Benten, Yasmin Behrang, Ludmilla Unrau, Victoria Weissmann, Gerrit Wolters-Eisfeld, Susanne Burdak-Rothkamm, Felix R. Stahl, Martin Anlauf, Patricia Grabowski, Markus Mobs, Jan Dieckhoff, Bence Sipos, Martina Fahl, Corinna Eggers, Daniel Perez, Maximillian Bockhorn, Jakob R. Izbicki, Ansgar W. Lohse, Jorg Schrader. Establishment of the First Well-differentiated Human Pancreatic Neuroendocrine Tumor Model. Mol Cancer Res 16, 496-507, 2018

1-14. (canceled)

15. A method of treating SSTR3 expressing tumors in a subject in need thereof, comprising administration of an effective amount of a compound of formula (I): 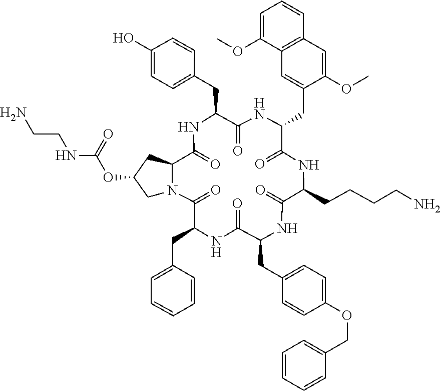
or a pharmaceutically acceptable salt and/or solvate thereof, alone or in combination with one or more physiologically acceptable excipients.

16. The method according to claim 15, wherein the SSTR3 expressing tumors are selected from non-functioning pituitary adenomas, neuroendocrine-related malignancies, including pancreatic tumors, pheochromocytomas, paragangliomas, lung carcinoids, and breast cancer.

17. The method according to claim 15, wherein the compound of formula (I), or the pharmaceutically acceptable salt and/or solvate thereof, is administered on a daily basis.

18. The method according to claim 15, wherein the subject is a human.

19. The method according to claim 15, wherein the compound of formula (I), or the pharmaceutically acceptable salt and/or solvate thereof is administered in an amount ranging from 0.5 to 5 mg/day.

20. The method according to claim 19, wherein the compound of formula (I), or the pharmaceutically acceptable salt and/or solvate thereof is administered in an amount ranging from 0.2 to 2.5 mg/day.

21. The method according to claim 20, wherein the compound of formula (I), or the pharmaceutically acceptable salt and/or solvate thereof is administered in an amount ranging from 0.1 to 2 mg/day.

22. The method according to claim 15, wherein the compound of formula (I), or the pharmaceutically acceptable salt and/or solvate thereof is administered in the form of a pharmaceutical composition comprising the compound of formula (I), or a pharmaceutically acceptable salt and/or solvate thereof together with at least one physiologically acceptable excipient.

23. The method according to claim 22, wherein the pharmaceutical composition is administered by oral, sublingual, rectal, intravascular, intravenous, or subcutaneous route.

24. The method according to claim 23, wherein the pharmaceutical composition is administered by intravenous route.

25. The method according to claim 22, wherein the pharmaceutical composition is in a solid or a liquid form.

26. The method according to claim 25, wherein the solid form is selected from a powder, a tablet, a granulate, an aggregate, a compressed or coated pill, and a hard or gelatine capsule.

27. The method according to claim 25, wherein the liquid form is a suspension, a syrup, or a liquid for injection.

28. The method according to previous claim 15, wherein the compound of formula (I) or the pharmaceutically acceptable salt and/or solvate thereof is administered to a patient in combination with at least one additional active principle.

29. The method according to claim 28, wherein the at least one additional active principle is selected from a secretagogue of the insulin, a promoter of the insulin secretion, an insulin sensitiser, a low insulin dose, an agent with dopamine receptor 2 agonism, an agent having anti-angiogenic effects, and a chemotherapeutic agent.

30. The method according to claim 29, wherein the agent with an insulin sensitiser activity is metformin, the agents with a secretagogues of the insulin are sulfonylureas or incretin-based drugs, including vindagliptin and nataglinide, the agents with a promoter of the insulin secretion are GLP-1 agonists, including liraglutide and exenatide, the agents with dopamine receptor 2 agonism are cabergoline or bromocriptine and the chemotherapeutic agent is temozolomide.

31. The method according to claim 28, wherein the compound of formula (I) or the pharmaceutically acceptable salt and/or solvate thereof and the at least one additional active principle are administered simultaneously, separately or sequentially.
